(19) United States
(12) Patent Application Publication
(10) Pub. No.: US 2026/0069237 A1
(43) Pub. Date: Mar. 12, 2026

(54) Wearable Soft Electronics-Based Stethoscope

(71) Applicant: Georgia Tech Research Corporation, Atlanta, GA (US)

(72) Inventors: Woon-Hong Yeo, Suwanee, GA (US); Min-Kyung Yeo, Daejeon (KR); Sung Hoon Lee, Atlanta, GA (US)

(21) Appl. No.: 18/707,766

(22) PCT Filed: Nov. 7, 2022
(86) PCT No.: PCT/US2022/049141

§ 371 Date: May 6, 2024

Related U.S. Application Data
(60) Provisional application No. 63/276,830, filed on Nov. 8, 2021.

(51) Int. Cl. A61B 7/04; A61B 5/00; A61B 5/08; G16H 40/67; H04R 1/46; H04R 3/06; H04R 19/04

(52) U.S. Cl. CPC A61B 7/04; A61B 5/0004; A61B 5/0816; A61B 5/7207; A61B 5/7225; A61B 5/725; A61B 5/7267; G16H 40/67; H04R 1/46; H04R 3/06; H04R 19/04; A61B 2560/0214; A61B 2562/028; A61B 2562/166; A61B 2562/227; H04R 2201/003

(57) ABSTRACT

An electronic stethoscope (100) for wearing on a body of a user includes a skin-mountable first circuit (110) includes a micro-electronic microphone (112) coupled thereto. The micro-electronic microphone (112) is configured to sense sounds from the body of the user (10) and to generate an analog signal representative thereof. A skin-mountable second circuit (130) is not contiguous with the first circuit (110), is spaced apart therefrom and includes circuitry that processes the analog signal from the electronic microphone (112). A flexible connector (140) electrically couples the first circuit (110) to the second circuit (130).

### CROSS-REFERENCE TO RELATED APPLICATION(S)

This application claims the benefit of U.S. Provisional Patent Application Ser. No. 63/276,830, filed Nov. 8, 2021, the entirety of which is hereby incorporated herein by reference.

### BACKGROUND OF THE INVENTION

### 1. Field of the Invention

The present invention relates to medical sensing devices and, more specifically, to a wearable stethoscope.

### 2. Description of the Related Art

Chronic obstructive pulmonary disease (COPD) and cardiovascular disease (CVD) are the leading causes of death worldwide, taking almost a million lives each year. COPD and CVD are umbrella terms for a group of diseases that cause heart and lungs to malfunction, restricting blood flow to cause difficulty breathing and severe discomfort. An alarming 80% of COPD deaths occur in low and middle-income countries (LMICs), where the lack of accessibility to healthcare treatment and affordability for current medical devices limits the feasibility of tracking the development of these progressive diseases over extended periods. For the diagnosis and care of patients with COPD and CVD, accurate auscultation is helpful for the diagnosis at an early stage and evaluation of the treatment response. Wheezing is important in the diagnosis and monitoring of those diseases. Crackles have a crucial significance in diagnosing pneumonia, idiopathic pulmonary fibrosis, and pulmonary edema. Stridor sounds suggest severe obstruction of the upper airway and is helpful for the patient's emergency treatment. Heart sound also provides significant information for diagnosing and identifying various valvular heart diseases and heart failures. If there is sound splitting in S1 and S2, some cardiovascular diseases such as valve diseases, arrhythmias, and pulmonary arterial hypertension can be suspected. If the pathological sound S3 or S4 sound is heard, it may suggest valve diseases, heart failure, and acute coronary diseases.

Auscultation has been the most basic and vital diagnostic method in the medical field because it is non-invasive, fast, informative, and inexpensive. Although many imaging and diagnostic technologies such as chest computed tomography and echocardiogram have been widely applied in clinical practice, auscultation is utilized as a primary diagnostic tool, especially in the LMICs. However, auscultation with conventional stethoscopes has fundamental limitations. Most stethoscopes cannot record the detected sounds, making it difficult to share the auscultatory sounds with other medical staff. In addition, analysis of auscultation sounds is quite different depending on the knowledge and experience of the clinician. As a result, some critical respiratory or heart diseases are underdiagnosed or misdiagnosed. Recently, the incidence and socioeconomic burden of COPD and CVD continue to increase due to the worsening aging population, air pollution, and various infectious diseases. Early diagnosis and accurate monitoring using exact auscultation are becoming more crucial and urgently required to improve auscultation technology.

Digital stethoscopes assist auscultation real-time and telemedicine diagnosis by recording and converting acoustic sound to electrical signals, amplifying subtle sounds inaudible using acoustic stethoscopes. They can be used instead of binaural stethoscopes in everyday patient care. In addition, these devices can be supplemented with computer software to improve diagnostic capabilities, though effective diagnosis using signal processing is still unavailable. Although signal graphs define quantitative measurements and reduce the subjectiveness of diagnosis from different physicians, varying positions, and pressure of stethoscope placement on the chest and the back still causes unwanted friction noise and human errors during data collection. This is especially a concern for patients self-operating a digital stethoscope at home, who lack experience compared to trained medical professionals. The rigidity and bulkiness of current digital stethoscopes create an unmet need for a more patient-friendly digital stethoscope. Furthermore, digital stethoscopes cannot detect pathology or aberrant sounds for diagnostic purposes. They just serve as sound recording devices, with the ability to graphically show the sound in the form of a phonocardiogram in some circumstances.

Many researchers desire computer algorithms to be accompanied for automated diagnosis. The approach in the vast majority of studies includes a single chest recording. However, with more sophisticated diagnoses, this is frequently insufficient. Multiple auscultation sites are required, which may lead to more errors.

Existing digital stethoscopes tend to be built around rigid materials and conventional electronic packaging, which can cause substantial air gaps and delamination on the curved the user's skin. This can result in unwanted artifacts and noise in the data stream from the digital stethoscope.

Therefore, there is a need for a less expensive, accurate, and wearable digital stethoscope device that allows for early cardiovascular and respiratory disease detection through continuous monitoring for use in telehealth and other forms of remote medical diagnosis.

### SUMMARY OF THE INVENTION

The disadvantages of the prior art are overcome by the present invention which, in one aspect, is an electronic stethoscope for wearing on a body of a user. A skin-mountable first circuit includes a micro-electronic microphone coupled thereto. The micro-electronic microphone is configured to sense sounds from the body of the user and to generate an analog signal representative thereof. A skin-mountable second circuit is not contiguous with the first circuit, is spaced apart therefrom and includes circuitry that processes the analog signal from the electronic microphone. A flexible connector electrically couples the first circuit to the second circuit.

In another aspect, the invention is a method of detecting a physiological phenomenon in a user having a body, in which a skin-wearable digital stethoscope is applied to the body of the user. User-generated sounds are sensed with digital stethoscope over a period of time and a digital signal representing the sounds is generated. The digital signal is transmitted to a remote device. A convolutional neural network running on the remote device is trained with digital representations of sounds that correspond to a plurality of physiological phenomena. The digital signal is applied to the convolutional neural network so as to generate an indication of a probability that the digital signal corresponds to one of the plurality of physiological phenomena. The probability is displayed.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a side view schematic diagram of one embodiment of a digital stethoscope.

FIG. 2 is a plan view of the embodiment shown in FIG. 1

FIG. 3 is a schematic diagram of digital stethoscope communicating with a remote device.

FIG. 4 is a flow chart showing one method of detecting a physiological phenomenon.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

One embodiment of a soft wearable stethoscope system for ambulatory cardiopulmonary auscultation uses a class of technologies with advanced electronics, flexible mechanics, and soft packaging that serves as a self-operable wearable for continuous cardiovascular and respiratory monitoring. The embodiment allows for accurate cardiorespiratory data collection in daily activities to diagnose various pulmonary abnormalities. Improving the signal-to-noise ratio from the wavelet-denoised sound collection minimizing circuitry makes the device more compact. Training a machine learning model to correctly identify stridor, rhonchi, wheezing, and crackling lung sounds from the device are functions of the embodiment. A user-friendly mobile device application can record heart and lung sounds, track and display real-time signals, automatically diagnose various abnormal lung sounds and upload information to a synchronized local memory remotely and securely.

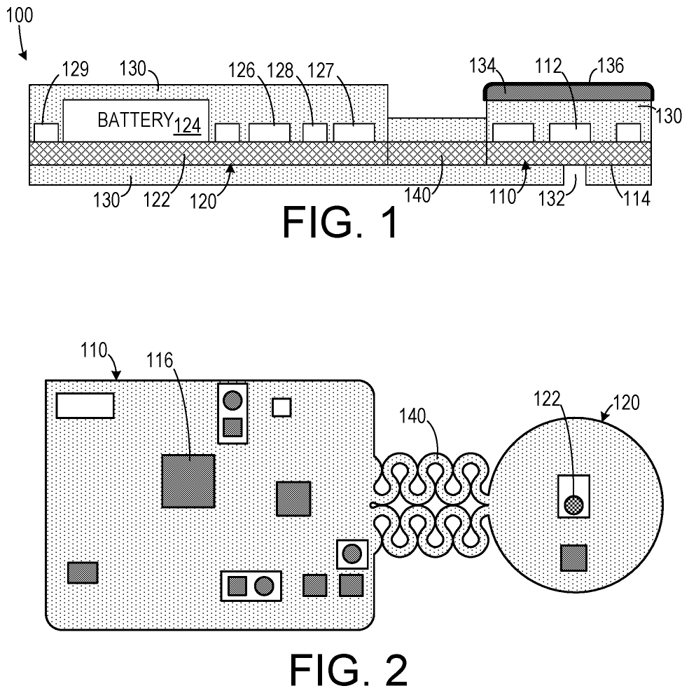

As shown in FIGS. 1 and 2, one embodiment of an digital electronic stethoscope 100 includes a skin-mountable first circuit 110 that includes a micro-electronic MEMs microphone 112 coupled thereto. The micro-electronic microphone 112 senses sounds from the user's body and generates an analog signal corresponding to the sounds. The first circuit 110 includes a first flexible printed circuit board 114. A skin-mountable second circuit 120 is not contiguous with the first circuit and is spaced apart therefrom. It includes circuitry that processes the analog signal from the electronic microphone 112 that is received through a flexible connector 140 which electrically couples the first circuit 110 to the second circuit 120. The flexible connector 140 has an undulated form factor that facilitates bending and expansion.

The first circuit 110 includes a first flexible printed circuit board 114 and the second circuit 120 includes a second flexible printed circuit board 122 that does not touch the first flexible printed circuit board 114. This isolates unwanted noises that can be generated through interaction between the second circuit 120 and the user's skin and clothing.

The first circuit 110, the second circuit 120 and the flexible connector 140 are encapsulated by a biocompatible elastomer envelope 130, which in one embodiment includes a medical grade silicone rubber. The elastomer envelope 130 defines a hole 132 under the microphone 112 to facilitate sound transmission therethrough. A tacky elastomer layer 134 and a fabric layer 136 is placed over the portion of the elastomer envelope 130 covering the first circuit 110 to reduce the amount of sound generated by the user's clothing near the microphone 112 due to the clothing sticking to the elastomer 130.

The second circuit 120 can include, for example, an analog-to-digital converter 128 that converts the analog signal from the micro-electronic microphone 112 into a digital representation of the analog signal, a micro-controller 126 that processes the digital representation and that can include a band pass filter for removing motion artifacts generated by the body of the user from the digital representation, and a low energy personal area network transmitter 129 (such as a Bluetooth-low-energy unit) that transmits data from the micro-controller 126 to a remote device, such as a computer or smart phone. A preamplifier 127 can amplify the signal from the micro-electronic microphone 112 and can be configured to filter out frequencies beyond at least one cut-off frequency from the signal. A rechargeable battery 124 powers the first circuit 110 and the second circuit 120.

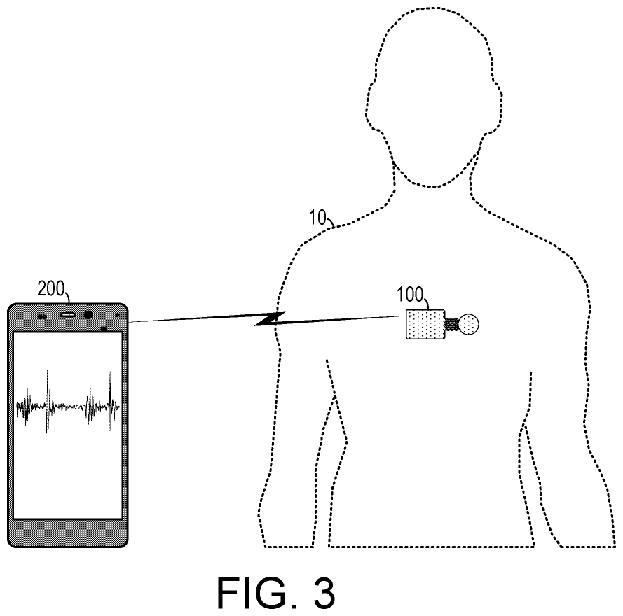

As shown in FIG. 3, the electronic stethoscope 100 is adhered to the chest of the user 10. (As will be readily understood, it can be adhered to other parts of the user's body to process sounds from those parts. For example, it could be applied to the user's knee to process sounds generated by the knee as part of a diagnostic process.) The data from the electronic stethoscope 100 can be transmitted to a remote device 200 for further processing.

The remote device can be programmed with a convolutional neural network that determines a probability that the data received from the personal area network transmitter correlates to an item from a data set with which the convolutional neural network has been trained. The item from the data set can correspond to a selected one of known physiological phenomena. For example, the known physiological phenomena can include lung sounds such as stridor, rhonchi, wheezing, and crackling lung sounds. It can also include different heartbeat sounds. The convolutional neural network can then be used to assist a diagnostician by relating the sensed sounds to corresponding diagnoses.

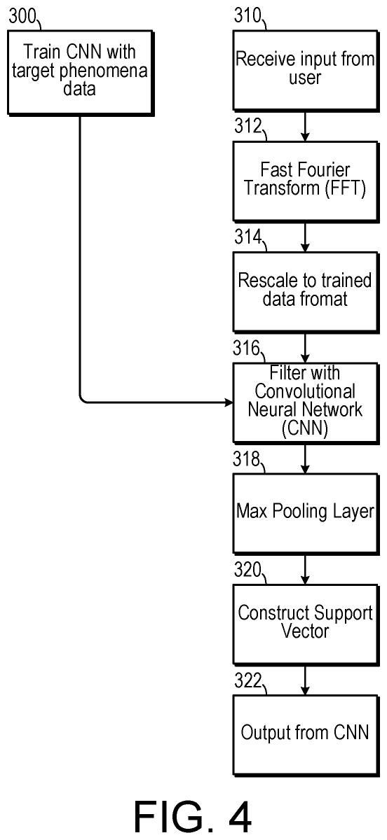

As shown in FIG. 4, one method of detecting a physiological phenomenon in a user having a body begins with training 300 the convolutional neural network (CNN) with data corresponding to different relevant sounds with labels for the types of sounds and, possibly, with diagnoses corresponding to the sounds. The skin-wearable digital stethoscope is applied to the body of the user and input is received from the stethoscope 310, which senses the sounds over a predetermined period of time and which transmits the corresponding to the remote device. A fast Fourier transform (FFT) can be applied 312 to the data to transform the data into a domain that is usable by the CNN. The transformed data can be rescaled 314 to fit the data format of the CNN and then the data is passed through the CNN 316, which determines a maximum pooling layer 318 and constructs a support vector 320 prior to generating an output 322 which can include an indication of which trained vector has the highest probability of corresponding to the received data.

One experimental embodiment of a soft wearable stethoscope employs nanomaterial printing, system integration, and soft material packaging to make a miniaturized, soft wearable stethoscope for a remote patient cardiopulmonary auscultation. The soft wearable stethoscope has an exceptionally small form factor and mechanically soft and flexible properties, allowing for intimate skin integration and self-operable auscultation for remote and continuous monitoring without physical interactions between patients and physicians. The soft mechanical characteristics include an elastomeric enclosure with an inner silicone-gel (300 μm in thickness and 4 kPa in Young's modulus. This arrangement employs the gentle placement of the device on the curved skin of the chest and the back via a thin, conductive hydrogel coupling layer to auscultate cardiac and respiratory activities. The silicone-gel backing provides reversible, multiple uses of the device with maintained sound detection qualities typically for at least two days.

This system uses a micro-electronic mechanical system (MEMS) microphone due to the small diaphragms for sound recording. Collected sounds from the microphone are then converted to digital signals through the analog-to-digital converter and streamed in real-time via the BLE chip for data processing. After sound collection, signal processing and denoising algorithm are used to filter out extraneous noise and label signals with various classes.

An important design point of the device is to isolate the microphone from the core circuit area, which provides an enhanced and more stable contact to the skin for noise-reduced continuous auscultation. The integrated soft wearable stethoscope can measure heart and lung sounds for more than 10 hours with continuous wireless data transmission. This device is powered by a miniaturized, rechargeable, lithium-ion polymer battery (40 mAh capacity). The battery's two terminals and the circuit's power pads are soldered with small neodymium magnets for a guided battery connection and continued uses.

Computational finite element analysis and corresponding experiments were used to capture the stretching and bending mechanics of the device on the human skin, mimicking the user's respiration cycles. When evaluated by a digital force gauge (EMS303, Mark-10) and a multimeter (DMM7510, Tektronix), the soft and flexible system, enclosed by low-modulus elastomers, displayed mechanical stability without fractures. Resistance fluctuation was negligible over the 100 cycles with less than 30 mΩ variations. The total resistance change was about 0.41 mΩ for cyclic stretching and 0.71 mΩ for cyclic bending tests.

In use, collected sounds through the app on the remote device can go through preprocessing, machine learning, and classification using convolutional neural networks (CNN). For instance, coarse breathing crackles during inhaling is a symptom of COPD, while the appearance of S3 and S4 heart signals can indicate cardiac dysfunction. Collectively, the fully portable soft wearable stethoscope can offer a unique opportunity for remote, digital health monitoring of patients without frequent visits to hospitals.

For high-quality, low-noise auscultation, it is important to maintain the intimate contact of the wearable microphone system to the skin, even with movements in daily life. The thin and flexible soft wearable stethoscope makes conformable contact with the skin. Furthermore, when the top enclosure is removed, the microphone island unit shows great skin contact with unnoticeable air gaps, providing high-quality sound recording.

Various types of daily activities have different sources of noise that can negatively affect the recording of sounds with a soft wearable stethoscope. The soft wearable stethoscope can successfully handle and control motion artifacts with the device form factor and maintained skin-contact quality.

Another important part of motion-artifact control is to ensure the minimized changes of air gaps between the skin and the diaphragm inside the microphone since the gap acts as an acoustic capacitance converting pressure wave to electrical signals. The soft device offers skin-conformal lamination to withstand any air gap changes during different activities, aided soft gel layers. The soft wearable stethoscope of the present invention has excellent skin contact, can minimize the air acoustic impedance between the epidermis and the diaphragm inside the microphone. Additional filtering of the first-level cut-off frequencies is used to remove the unwanted high-frequency noise, typically caused by motion, speech sounds, and beeping sounds in clinical settings.

Wavelet transformation on heart and lung sound signals and noise filtering processes are crucial in this study since the microphone captures all sounds from the body and the surrounding. Wavelet denoising using a threshold algorithm is one of the most powerful methods for suppressing noise in digital signals. In auscultation, determining threshold values for heart and lung sounds is critical in the wavelet threshold denoising method. A modest threshold value may not eliminate all the noisy coefficients, while significant thresholding sets more coefficients to zero, removing features from the decomposed data. The experimental embodiment used two parts of filter banks to denoise the surrounding noise for auscultated data, including the analysis filter and the synthesis filter bank. The analysis filters decompose the inputted heart and lung sounds into down-sampled sub-bands, and the synthesis filter bank reconstructs the original heart and lung sound data after up-sampling. After an audio signal is read through the algorithm, it adds Gaussian noise to the raw signal to form a noisy signal. After the SNR and the root mean square error (RMSE) is calculated for the noisy signal, the threshold value for wavelet thresholding is calculated by SNR over RMSE value, also depending on the noise intensity and the decomposition stage. This thresholding is applied to decomposed wavelet coefficients, and a soft threshold is used for lung auscultation. The soft thresholding provides a consistent difference between the reconstructed and the original signals, causing sharp sounds to be smoothed. The last step is to reconstruct the lung sound signals leveraging the soft-threshold wavelet coefficients fed into the synthesis filter bank.

When necessary, physicians or clinicians use conventional digital stethoscopes to collect sound signals from patients. However this way includes an inherent problem of subjective auscultation analysis of collected sounds. For example, pulmonologists who diagnose and treat respiratory diseases may provide different diagnostic opinions, depending on the quality or duration of recorded sounds. Here, the soft wearable stethoscope of the present invention has a significant advantage with the capabilities of noise-controlled continuous, real-time recording of high-quality sounds, quantitative data analysis, and automated objective classification of diseases based on machine learning (e.g., lung abnormalities like crackle, rhonchi, wheeze, and stridor). The data are divided into training and test sets using a 75-25 percent split, ensuring that no training and test sets overlapped. This test is repeated four times as part of five-fold cross-validation. Each sample is clustered into 2-second packets and fed into CNN-based machine learning. To additionally show wavelet transformed heart sounds, 20-second heart sound data also were analyzed with derived heart rate for further application.

The advantages of the soft wearable stethoscope in terms of the form factor, portability, and high-quality sound recording offer the potential for applications such as sleep studies. The soft device, mounted on the chest, successfully measures and collects snoring sounds separated frequency ranges from heart sounds. Sleep-disordered breathing, such as snoring, is linked to cardiovascular illness, including heart failure, hypertension, and increased arrhythmias. The time of snoring in relation to the inspiration period would reveal the anatomical origin of snoring: tongue or soft palate during inhale or exhale, according to scientific explanation. Compared to soft palate snoring, tongue snoring reveals uneven timing relative to the breathing cycle and inconsistent frequency ranges from spectrograms, indicating obstructive sleep apnea that needs to be screened for treatment. Furthermore, snoring has been linked to respiratory symptoms, including wheezing and chronic bronchitis. Those with asthma and sleep-disordered breathing have poorer sleep quality and decreased nocturnal oxygen saturation. Tongue inhale has a distinct range of power in frequency from 0 Hz to 500 Hz as well as distinct peaks ranging from 500 Hz to 1 kHz, followed by decreasing power of the signal in exhaling. On the other hand, tongue snoring during exhale shows a gradual increase of power from the inhale ranging up to 250 Hz, capturing distinct signal peaks. This measurement also presents the palatal snoring during the inhale. Compared to tongue snoring, similar signal power is shown throughout the range of the frequency during inhale except the range of 350˜400 Hz. Overall, the experimental embodiment demonstrated the soft device's potential for more accurate at-home sleep monitoring by simultaneously monitoring cardiopulmonary sounds and electrophysiological signals.

In making one embodiment of the electronic stethoscope of the present invention, the soft elastomer gel, which in one embodiment is a silicon rubber (e.g., Ecoflex, available from Smooth-On, Macungie, PA) was used as a base adhesion layer for the soft wearable stethoscope. A mixture of the gel was spin-coated to form a thin layer, and the integrated circuit was placed on top of the gel layer. A silicone gel (High-Tack Silicone Gel, Factor II) was used on a fabric layer (3M 9907T). The fabric was cut out in a circle shape on top of the encapsulated microphone island for better pressure applied on the microphone.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps.

Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. An electronic stethoscope for wearing on a body of a user, comprising:
(a) a skin-mountable first circuit that includes a micro-electronic microphone coupled thereto, the micro-electronic microphone configured to sense sounds from the body of the user and to generate an analog signal representative thereof;
(b) a skin-mountable second circuit that is not contiguous with the first circuit and spaced apart therefrom, that includes circuitry that processes the analog signal from the electronic microphone; and
(c) a flexible connector that electrically couples the first circuit to the second circuit.

2. The electronic stethoscope of claim 1, wherein the first circuit comprises a first flexible printed circuit board and wherein the second circuit comprises a second flexible printed circuit board.

3. The electronic stethoscope of claim 1, wherein the first circuit, the second circuit and the flexible connector are encapsulated by a biocompatible elastomer envelope.

4. The electronic stethoscope of claim 3, wherein the biocompatible elastomer envelope comprises a medical grade silicone rubber.

5. The electronic stethoscope of claim 3, wherein the biocompatible elastomer envelope defines a hole on one side disposed adjacent to the micro-electronic microphone to allow sound transmission therethrough.

6. The electronic stethoscope of claim 1, wherein flexible connector has an undulated form factor.

7. The electronic stethoscope of claim 1, wherein the second circuit comprises:
(a) an analog-to-digital converter that converts the analog signal from the micro-electronic microphone into a digital representation thereof; and
(b) a micro-controller that processes the digital representation.

8. The electronic stethoscope of claim 7, wherein the second circuit includes a band pass filter for removing motion artifacts generated by the body of the user from the digital representation.

9. The electronic stethoscope of claim 7, further comprising:
(a) a low energy personal area network transmitter, coupled to the second circuit, that transmits data from the micro-controller; and
(b) a remote device that receives the data from the low energy personal area network transmitter and that processes the data.

10. The electronic stethoscope of claim 9, wherein the remote device is programmed with a convolutional neural network that determines a probability that the data received from the personal area network transmitter correlates to an item from a data set with which the convolutional neural network has been trained.

11. The electronic stethoscope of claim 10, wherein the item from the data set corresponds to a selected one of known physiological phenomena.

12. The electronic stethoscope of claim 10, wherein the selected one of the known physiological phenomena includes a phenomenon selected from a list consisting of: stridor, rhonchi, wheezing, crackling lung sounds, and combinations thereof.

13. The electronic stethoscope of claim 1, wherein the second circuit comprises a preamplifier that amplifies a signal from the micro-electronic microphone and that filters out frequencies beyond at least one cut-off frequency therefrom.

14. The electronic stethoscope of claim 1, wherein the second circuit includes a rechargeable battery that powers the first circuit and the second circuit.

15. The electronic stethoscope of claim 1, further comprising a fabric layer adhered to an outside side of the first circuit and configured to prevent a clothing item worn by a user from sticking to the first circuit.

16. The electronic stethoscope of claim 1, further comprising a fabric layer adhered to an outside side of the first circuit and configured to prevent a clothing item worn by the user from sticking to the first circuit.

17. A method of detecting a physiological phenomenon in a user having a body, comprising the steps of:
(a) applying a skin-wearable digital stethoscope to the body of the user;
(b) sensing user-generated sounds with digital stethoscope over a period of time and generating a digital signal representing the sounds;
(c) transmitting the digital signal to a remote device;
(d) training a convolutional neural network running on the remote device with digital representations of sounds that correspond to a plurality of physiological phenomena;
(e) applying the digital signal to the convolutional neural network so as to generate an indication of a probability that the digital signal corresponds to one of the plurality of physiological phenomena; and
(f) displaying the probability.

18. The method of claim 17, further comprising the step of filtering digital signal to remove user motion artifacts generated by the body of the user.

19. The method of claim 17, wherein the physiological phenomena include at least one phenomenon selected from a list consisting of: stridor, rhonchi, wheezing, crackling lung sounds, and combinations thereof.

20. The method of claim 17, wherein the skin-wearable digital stethoscope includes:
(a) a skin-mountable first circuit that includes a micro-electronic microphone coupled thereto, the micro-electronic microphone configured to sense sounds from the body of the user and to generate an analog signal representative thereof;
(b) a skin-mountable second circuit that is not contiguous with the first circuit and spaced apart therefrom, that includes circuitry that processes the analog signal from the electronic microphone; and
(c) a flexible connector that electrically couples the first circuit to the second circuit.
